(19) United States
(12) Patent Application Publication
(10) Pub. No.: US 2009/0312560 A1
(43) Pub. Date: Dec. 17, 2009

(54) PROCESSES FOR MAKING PREGABALIN AND INTERMEDIATES THEREFOR

(76) Inventor: Lambertus Thijs, Wijchen (NL)

Correspondence Address: SYNTHON IP INC, 7130 HERITAGE VILLAGE PLAZA, STE 202, GAINESVILLE, VA, 20155, US

(21) Appl. No.: 12/481,173

(22) Filed: Jun. 9, 2009

Related U.S. Application Data
(60) Provisional application No. 61/060,350, filed on Jun. 10, 2008.

(51) Int. Cl. C07D 207/12; C07C 69/34; C07C 53/126

(52) U.S. Cl. 548/531; 560/196; 562/512

(57) ABSTRACT

Processes for making a diester compound of formula (B) and for converting it to pregabalin, especially via a compound of formula (2), can provide several advantages.

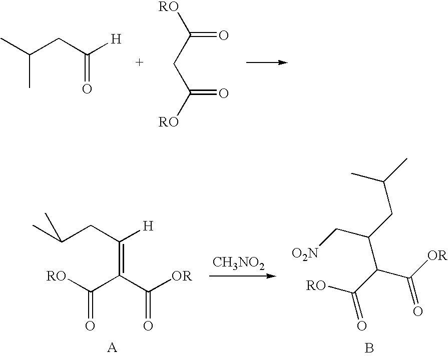

The compound (B) can be converted to the compound (2) via reductive hydrogenation.

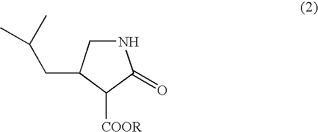

This application claims the benefit of priority under 35 U.S.C. § 119(e) from prior U.S. Provisional Application Ser. No. 61/060,350, filed Jun. 10, 2008, the entire contents of which are incorporated herein by reference.

### BACKGROUND OF THE INVENTION

The present invention relates to intermediates and processes useful for making 4-amino-3-(2-methylpropyl)butanoic acid, an important pharmaceutical.

Pregabalin, S—(+)-4-amino-3-(2-methylpropyl)butanoic acid of the formula (1A)

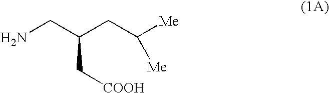

is a structural analogue of GABA. Pregabalin acts as a modulator of voltage-gated calcium channels in the CNS, having the potential to treat neuropsychiatric disorders and pain. It is currently sold as a capsule for oral administration under the brand name LYRICA® by Pfizer.

Pregabalin is an amino acid, i.e., it contains both a basic amino group and an acidic carboxy group, and thus can exist as a zwitterion (i.e., in a state where the carboxyl group is deprotonated and the primary amine moiety is protonated). Pregabalin may therefore form salts with both acids and bases. The pKa values are 4.2 and 10.6, respectively. In the marketed compositions, pregabalin is used in its free form.

Pregabalin is a single enantiomer of 4-amino-3-(2-methylpropyl)butanoic acid of the formula (1).

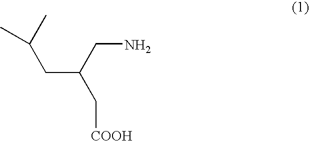

Due to the presence of one asymmetric center, a compound of this formula is obtainable both as a racemate (whereby it can be subsequently resolved into enantiomers, e.g., by a process suggested in WO 96/40617 (which is also published as U.S. Pat. No. 5,637,767)) or as the desired single enantiomer by an enantio-selective synthesis.

Racemic compound (1) was disclosed and prepared by Andruszkiewicz and Silverman in Synthesis 1989, p. 953. The single (S)-enantiomer, i.e., pregabalin, was later disclosed in WO 93/23383.

Various processes have been published for making the compound of the general formula (1), both racemate and single enantiomer. A brief review of some of them is provided in Drugs of the Future Vol. 24, No. 8, 1999, pp. 862-870. The last transformation step(s) of many of the known synthetic approaches are represented below in Scheme I, wherein for simplicity the actual conformation of starting and ending product in the respective articles and/or patent documents was set aside.

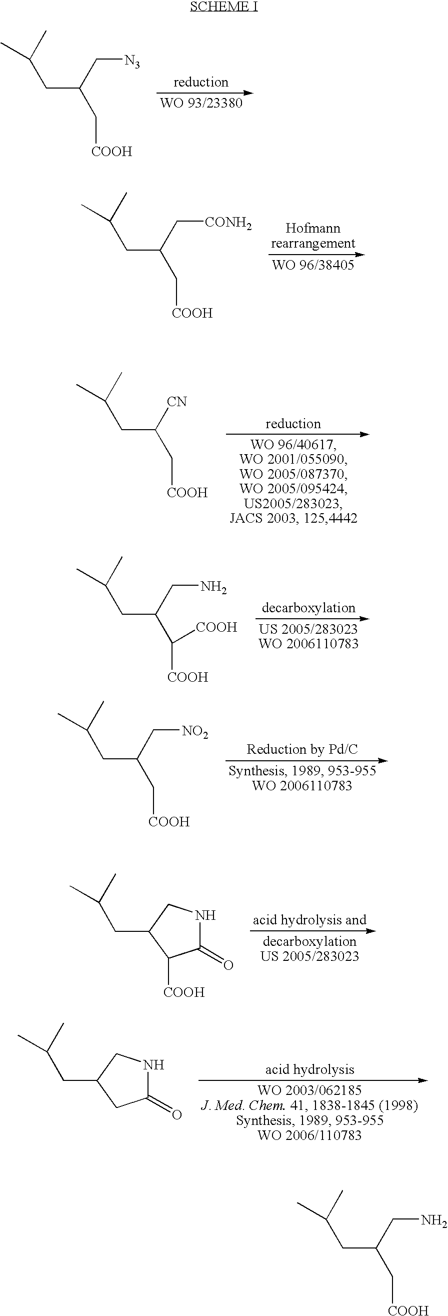

One of the interesting processes uses a 3-carboxy- and/or 3-carbalkoxy-4-isobutylpyrrolid-2-one of the formula (2a) or (2), respectively.

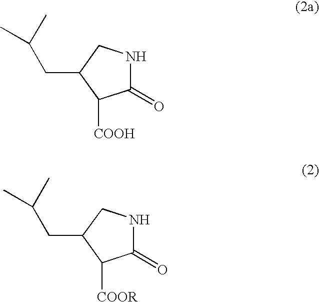

In the formula (2), R represents, inter alia, an alkyl group.

Two known processes for making the compound of the formula (2a) or (2) are as follows:

A) US 2005/283023 introduces a process in which a cyanodiester D is resolved into enantiomers by an enzymatic resolution using a lipase enzyme. The obtained (S)-cyano-ester-acid E is then further: a) reduced and decarboxylated; b) decarboxylated and reduced; or c) saponified, reduced and decarboxylated. In the first case, (a), the reaction proceeds as follows:

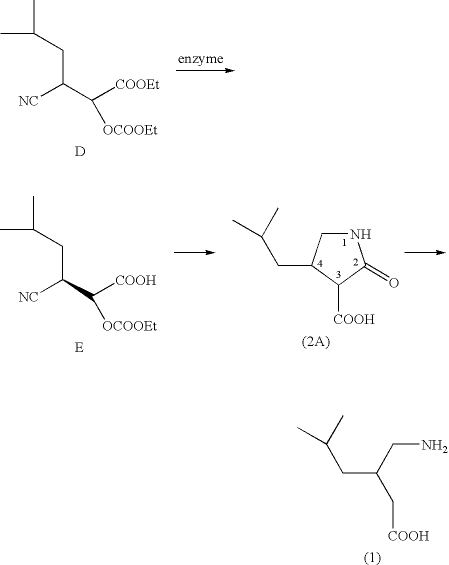

The final compound (1) is obtained in the desired (S)-conformation. The starting cyanodiester D is prepared by a multistep procedure, one of these steps being an addition of hydrogen cyanide on a double bond of a suitable precursor. A disadvantage of this process is the use of this toxic cyanide reagent.

B) WO 2006/110783 discloses several processes for preparing (S)-pregabalin starting from (S)-nitro-diester compounds (i.e., the compound (15) of the document, R1 and R2 are independently hydrogen, an alkyl group, a benzyl group, an aryl group, etc.) including the following scheme:

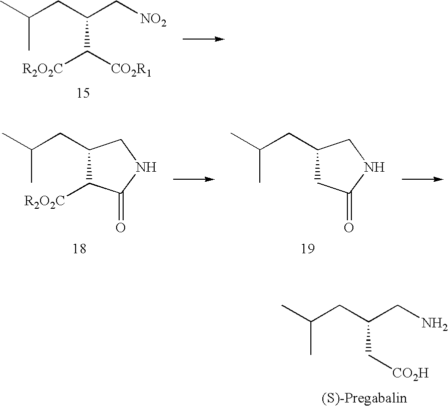

The diester is subjected to reductive cyclization to form the ester (18), which can correspond to formula (2) herein, in the (S) configuration. The disclosed reductive cyclization of (15) to (18) is achieved by a reduction with metal hydride in the presence of a Ni salt. Then the ester compound (18) (R is not H) is hydrolyzed by a base to an acid compound (18) (R═H, or compound (2a) herein in the S-configuration) and then decarboxylated to (19) by heating in an inert solvent to over 100° C. The final opening of the lactam ring of (19) is achieved by heating in inorganic acid.

The starting diester compound is taught to be prepared via a general process disclosed by Li H. et al. in JACS 2004, 126, 9906. This process, although not specifically disclosed for making the compound (15), involves an addition reaction of dialkylmalonate on a nitroalkene in the presence of certain cinchona-based catalysts which work with a high degree of enantioselectivity. The nitroalkene can be made by a complicated process involving an addition of nitromethane to the corresponding aldehyde, which yields a nitroaldol, which is then decomposed by trifluoroacetic anhydride (Scott E. Denmark and Lawrence R. Marcin, J. Org. Chem., 1993, 58, 3850). Thus, in essence, the suggested synthetic approach in WO2006/110783 is based on the following sequence

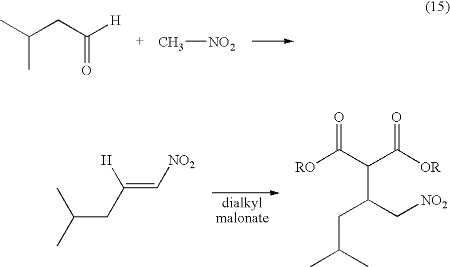

As the compound of the general formula (2) is a valuable intermediate for making the pharmaceutically useful compound of formula (1), it is desirable to provide an improved process for making it and for making pregabalin in general.

### SUMMARY OF THE INVENTION

The present invention relates to the discovery of an improved process for making a diester of formula (B) and to processes of converting the diester into pregabalin, including in particular via a compound (2). Accordingly, a first aspect of the invention relates to a process which comprises: (i) reacting isovaleraldehyde with an excess of a dialkyl malonate of the formula (10)

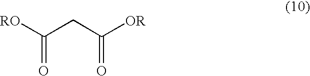

wherein R is a C1-C4 alkyl group, in the presence of a base reagent having a combination of a secondary amine and a carboxylic acid, to obtain a compound of formula (A)

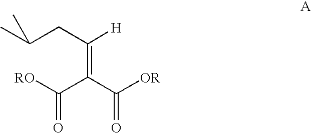

and (ii) reacting the compound of formula (A) with nitromethane, preferably with 1-1.5 molar equivalent thereof, under conditions of Michael addition, to yield the compound of formula (B)

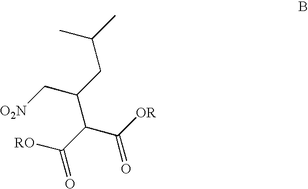

(as a racemate and/or a single enantiomer thereof) wherein R is as previously defined. The diester compound of formula (B) corresponds in principle to the formula (15) of WO2006/110783. The base regent can be a mixture of compounds, e.g., an acid and a base, but more conveniently is a single compound having both functionalities, namely an iminoacid such as proline. The compound (B) can be obtained in good yields and/or economy by the present invention and can be converted to pregabalin by known routes; e.g., via a compound of formula (2).

A second aspect of the invention relates to a process which comprises subjecting a compound of formula (B)

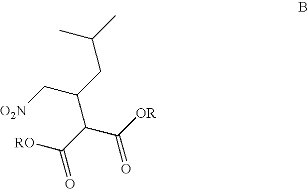

to reductive cyclization by hydrogen to form a compound of formula (2)

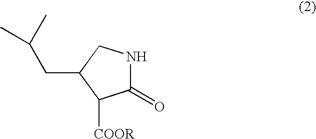

wherein R is a C1-C4 alkyl group, preferably methyl or ethyl group, and wherein the reductive cyclization is carried out in the presence of Raney-nickel and preferably under greater than atmospheric pressure of hydrogen. The compound of formula (2) can be converted to pregabalin by a variety of schemes. If the process resulting in (2) is not enantioselective, then the desired (3S),(4S) diastereomer can be resolved and then converted to pregabalin. A preferred conversion technique includes treating the (3S),(4S)-diastereomer of the compound (2) with a strong acid, e.g., HCl, under reactive conditions to form pregabalin.

A further aspect of the invention relates to a process for resolution, which comprises reacting a mixture which contains (3S),(4S) and (3R),(4R) diastereomers of the trans-compound of the formula (2)

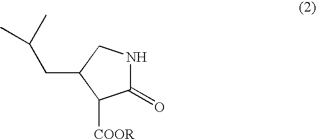

wherein R is a C1-C4 alkyl group, with an enzyme to obtain an enriched (3S),(4S)-diastereomer-containing mixture of the compound of formula (2). A preferred enzyme is pig liver esterase.

An additional aspect of the invention relates to a process for determining the conclusion of a reaction, which comprises: (a) subjecting a compound of formula (B)

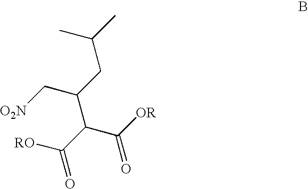

to reductive cyclization by hydrogen to form a compound of formula (2)

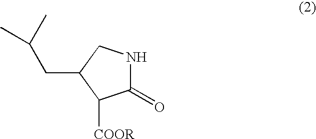

wherein R is a C1-C4 alkyl group, preferably methyl or ethyl group, and wherein the reductive cyclization is carried out in the presence of a hydrogen catalyst and preferably under greater than atmospheric pressure of hydrogen; (b) monitoring the reaction for the presence of the compound of formula (5)

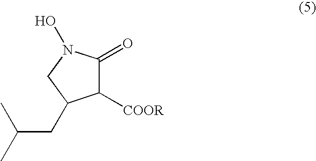

wherein R is a C1-C4 alkyl; and (c) terminating the reaction when the content of (5) is less than a predetermined amount, preferably less than 5%.

An additional aspect relates to a compound of formula (2)

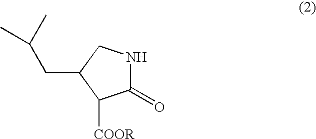

in a crystalline form, wherein R is a C1-C4 alkyl group, preferably methyl or ethyl group. Correspondingly, a further aspect of the invention relates to a process for precipitating the compound (2), which comprises contacting a solution which contains a compound of formula (2)

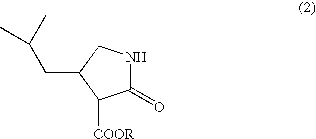

wherein R is a C1-C4 alkyl, with an antisolvent to precipitate said compound of formula (2) as a solid, preferably crystalline material.

Another aspect of the invention relates to a compound of formula (5)

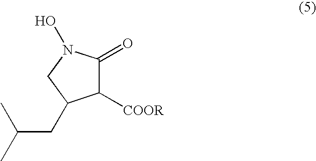

wherein R is a C1-C4 alkyl group, in an isolated form having a purity of at least 60%, preferably at least 80%. Such an isolated form of compound (5) is useful, inter alia, as a reference marker or standard.

### DETAILED DESCRIPTION OF THE INVENTION

The present invention relates to the discovery of useful processes for making a diester compound of formula (B) and to converting it to pregabalin, especially via a compound of formula (2). The first process of the invention can provide an improvement in making the compound of the general formula (B) and is illustrated below:

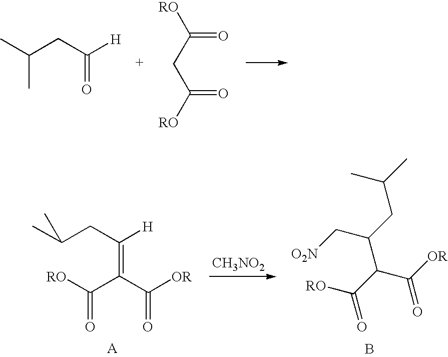

This reaction sequence is in contrast to the process suggested in WO 2006/110783 which would be generally represented as follows:

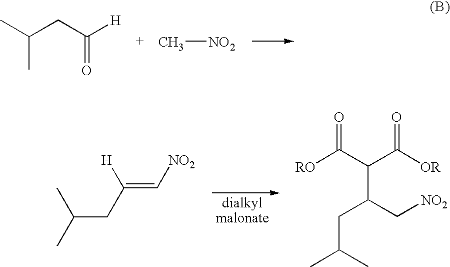

The process sequence according to the invention can provide higher overall yields than this prior suggested process and does not require decomposition agents.

For simplicity and convenience, all compound formulas used herein which do not specify a single enantiomer, such as formula (B), embrace racemic or mixed enantiomers as well as enriched or relatively pure individual enantiomers (i.e., relatively free of the other enantiomer), unless otherwise specified.

In the first step of the reaction sequence of the present invention, isovaleraldehyde reacts with dialkylmalonate (R is C1-C4 alkyl group and preferably is methyl or ethyl group) to yield the en-diester compound of formula A. The dialkylmalonate is normally used in excess, typically about 2 molar equivalents relative to the isovaleraldehyde. Both components react in an inert solvent, which is preferably a dipolar aprotic solvent (dimethyl sulfoxide, dimethyl formamide), hydrocarbon (e.g., hexane, heptane, cyclohexane, benzene, toluene, xylene, petroleum ether) or a highly boiling ether (e.g., methyl-tert.butyl ether), under a presence of a base reagent. The selection of a proper base is quite important as the condensation between the aldehyde and dialkylmalonate could yield also an isomeric beta-gamma unsaturated compound (A2) which, in the next step, would provide the unwanted isomer of the nitro-compound (B2) as an undesired impurity as shown below.

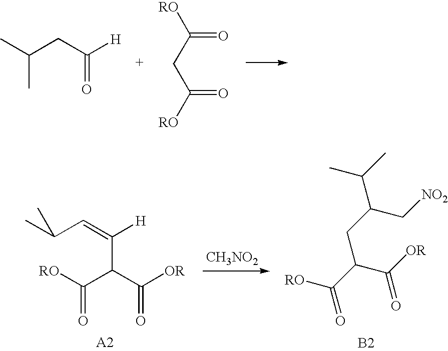

Another impurity may be a “dimer” (bis-dialkylmalonate compound), formed by the reaction of the excess of the dialkylmalonate with the formed compound (A) or (A2). A strong base could also induce a self-condensation of the aldehyde.

The base used in the condensation reaction therefore should be sufficiently weak to minimize the enolization of the aldehyde, but strong enough to activate the methylene group in the malonic diester. From this aspect, a good option is to use a base reagent having a mixture of a secondary aminic base with a carboxylic acid. This mixture can be obtained by combining two or more compounds to form the base reagent or more preferably can be obtained by a single compound having both functional groups. A particularly useful option for the present invention is to use an iminoacid as the base reagent, especially proline or similar iminoacids such as arginine.

The product of the formula (A) may be isolated from the reaction mixture, e.g., by diluting with a water immiscible solvent, extracting the water soluble parts with water, and evaporating the organic phase to dryness. Using the above conditions, an isolated product (A) comprising less than 10%, preferably less than 5% and more preferably less than 2% of the isomer (A2) is obtained. Preferably, the R group in the compound (A) is methyl group.

It is normally preferred that any excess of the dialkylmalonate remaining should be well removed from the product otherwise it could react in a further step. A suitable way to remove the unreacted dialkylmalonate is by fractional distillation of the crude product in vacuo. The dialkylmalonate is concentrated in the low boiling fractions, which are then removed. As a result, the content of the dialkylmalonate in the product can drop to less than 0.5%. Thus, an isolated form of the compound (A), meaning generally the product (A) essentially free from the solvent, in which the content of dialkylmalonate is less than 0.5% is a preferred form. Typically the R group in the compound (A) is methyl or ethyl.

In the second step, the compound (A), wherein R is C1-C4 alkyl group, preferably the isolated form thereof, and most preferably the isolated form with the minimized amounts of the side products and remainder of dialkylmalonate as specified above, reacts with nitromethane under conditions of Michael addition reaction. It is an advantage of this approach that the toxic nitromethane may be used only in a slight molar excess, e.g., 1-1.5 molar equivalent, contrary to the known cases where the nitromethyl moiety has been introduced to other substrates within a pregabalin synthesis (e.g., Anduszkiewicz and Silverman, Synthesis 1989, 953). The reduction in amount of nitromethane is probably possible because the two ester groups sufficiently activate the beta-position of the endiester (A) so that a larger excess of the nitromethane reagent is not necessary.

Advantageously, the reaction proceeds in the presence of a catalytic amount of a base, preferably a non-nucleophilic base e.g., 8-diazabicyclo[5.4.0]undec-7-ene (DBU), 1,4-diazabicyclo[2.2.2]octane (DABCO), 1,5-diazabicyclo(4:3:0)non-5-ene (DBN), 4-dimethyl-aminopyridine (DMAP). Because the base serves as a true catalyst it is not consumed in the reaction and thus can be present in sub-equivalent amounts. Hence less than stoichiometric amounts of base are typically used as the catalyst.

The reaction proceeds in an inert, preferably non-aqueous, solvent, which may be a C5-C10 hydrocarbon (e.g., hexane, heptane, cyclohexane, petroleum ether, benzene, toluene etc.), an ether, a chlorinated hydrocarbon, a dipolar aprotic solvent, etc. The reaction temperature may vary from ambient to reflux temperature. The product may be isolated from the reaction mixture after substantial removal of the remainder of starting materials and the base (e.g., by an extraction with an aqueous acid) by conventional means.

The isolated compound (B) may be obtained in a purity higher than 98% and may be further purified, if necessary, e.g., by a chromatographic procedure.

The diester (B) can be converted to pregabalin via many known routes. The preferred route involves forming the lactam compound of formula (2). The reductive cyclization can be carried out quite easily and by a variety of routes. Indeed, a repetition of the non-cyclizing reduction of the compound (B) in Example 1 of WO 2006/110783 did not yield the expected compound (3)

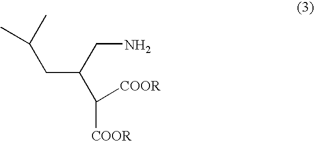

(=compound (16) of WO 2006/110873, R=ethyl, as a single enantiomer). The compound is even not detectable in the reaction mixture after the completion of hydrogenation reaction. Instead, the process yields the lactam of the formula (2). Apparently the compound (3) is so unstable that it forms easily the lactam (2) by splitting the R—OH moiety under the reduction reaction conditions. Thus a variety of mere reductive conditions can form the compound (2) from the diester compound (B).

In general, however, more conventional reductive cyclization conditions are used. The basic conditions of the reductive cyclization of gamma-nitroalkylmalondiesters forming the analogues of the compound (2) are known from Bull. Soc. Chim. Fr. 1962, 598 by Cologne J. et al. and from other sources. In general these conditions use hydrogenation (e.g., hydrogen gas or other hydrogen source) in the presence of a hydrogen catalyst.

It has been discovered that certain reductive hydrogenation conditions are more preferred than others. For example, the reduction process, particularly in larger scale and using hydrogenation with palladium or platinum catalyst, yields an impurity of the formula (5)

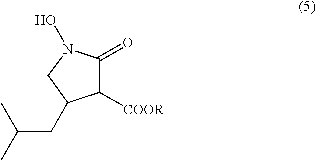

This impurity, the amount of which may raise up to 50% of the isolated reaction product under certain reaction conditions, is quite stable and it could be converted into the desired product (2) by “over” hydrogenation, i.e., only after hydrogenation for quite a long time or at an enhanced temperature and pressure. Therefore, its formation should be suppressed or the reaction conditions should allow to convert it into the desired (2).

Without wishing to be bound by any theory, it is assumed that the reduction of the nitro-malonate (B) proceeds according to the following scheme

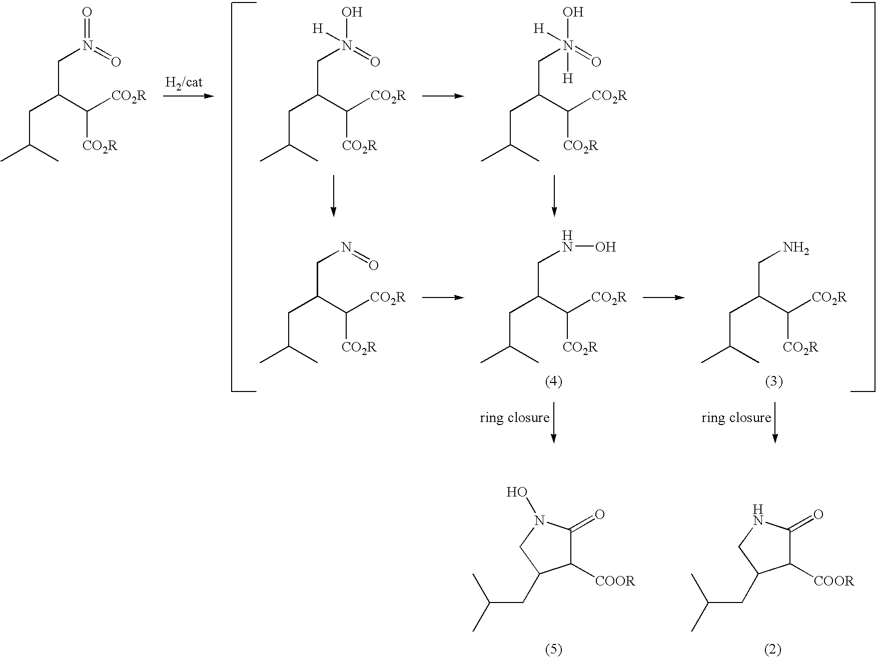

Fortunately, it was found that an activated nickel, e.g., Raney-nickel, catalyst is a very selective catalyst, which provides the lactam compound of the formula (2) only with minor amounts of the hydroxy-lactam impurity of the formula (5). It is able even to convert the mixtures of the compounds (2) and (5), e.g., reaction mixtures obtainable after reductive cyclization of (B) by hydrogenation on palladium or platinum catalysts, into essentially pure compound (2).

The hydrogenation of the compound (2) in the presence of Raney-nickel preferably proceeds with an enhanced pressure of hydrogen, typically 1.2-5 atmospheres. In an advantageous mode, the reaction can be carried out under such conditions for a sufficiently long time to achieve or ensure low amounts of compound (5) in the final product. The “sufficient time” may be determined by monitoring the process of hydrogenation (preferably on Ra—Ni) for the presence of the compound (5) and stopping the reaction not only after the starting material has been substantially or completely consumed, but also when the amount of the compound (5) is less than a pre-determined amount such as less than 5%, preferably less than 1%.

To facilitate monitoring the reaction mixture, it is often advantageous to prepare a reference material of the compound (5); that is a sample of an isolated form of the compound (5) with a defined content thereof, particularly higher than 60% and more particularly higher than 80%. This isolated or reference sample/marker of compound (5) can be analyzed by a suitable method (HPLC, TLC, etc.) to provide a standard analytical response (e.g., a response factor, a residence time, etc.). Samples from the reaction mixture can then be analyzed by the same method and compared to the response of the compound (5) to determine the content thereof. Accordingly, the above defined isolated form of the compound (5) forms a specific aspect of the present invention.

The reaction temperature for the above hydrogenation process is essentially ambient, but may vary from about 20° C. to 50° C. The convenient reaction solvent is a C1-C4 alcohol, preferably the alcohol with the same alkyl chain as the R group of the starting ester (B) to eliminate undesired trans-esterification.

In a known way of elaboration of the reaction mixture after hydrogenation, i.e., by removal of the catalyst by filtration and evaporation of the solvent, the desired crude (2) is generally obtained as an oil. It is possible to use this oil in the next reaction steps, however, it would be useful for many reasons to convert it into a solid material. It has been discovered that the solid state compound of the formula (2) may be obtained by precipitation thereof from the reaction mixture, concentrated reaction mixture or evaporated reaction mixture, by contacting the solution thereof with a suitable antisolvent. The “antisolvent” is a liquid, in which the compound (2) is insoluble or only very sparingly soluble. The suitable antisolvent includes a dialkylether, such as di-isopropyl ether or methyl isopropylether; a C5-C1 hydrocarbon, such as hexane, heptane, benzene, toluene; and mixtures thereof. The temperature of precipitation is ambient or lower than ambient. The precipitation in solid state is also a useful tool for purification of the compound (2).

The precipitation provides the solid, preferably crystalline form of the compound of formula (2), in a high degree of chemical purity, e.g., higher than 95%.

It should be also noted that the reductive cyclization of the nitrodicarboxylate (B) by hydrogen in the presence of a hydrogenation catalyst provides the lactam (2) (and the hydroxy-lactam (5) as well, if present) in the trans-configuration of the substituents in the position 3 and 4. If the starting nitro-compound (B) is a racemate, then a trans-racemate, i.e., a mixture of (3R,4R) and (3S,4S) diastereomers, of the compound of formula (2) is formed. If the starting compound (B) is a single enantiomer, then a single diastereomer, e.g., the preferred (3S,4S) diastereomer of the compound of formula (2) is formed. This orientation is desired as it yields directly the (S)-enantiomer of pregabalin by subsequent hydrolysis and cleavage.

The single trans-diastereomer of the compound (2) in the desired (3S),(4S) orientation may be obtained by the above process, if the starting compound (B) is obtained as a single (S) enantiomer, e.g., by an enatioselective addition of the nitromethane on the compound (A), wherein the base catalyst is a chiral base.

In an alternate and preferred way, which forms a specific aspect of the present invention, the single trans-diastereomer of (2) may also be obtained by resolution of the above trans-racemate. It was found that a useful resolution process comprises a reaction of the trans-racemate of the formula (2) with a suitable enzyme. A screen for the proper enzyme was performed and it was found that a highly selective enzyme is a pig liver esterase (PLE), which selectively hydrolyzes the ester group of the undesired 3R position, while the ester group in 3S position is maintained intact. Thus, the desired single (3S),(4S) diastereomer of the compound (2) is obtained as an ester, while the (3R),(4R) diastereomer is converted into an acid, thereby enriching the content of the (3S),(4S) ester. The acid can, and practically almost always is, separated from the ester compound such as by differences in solubility, etc., to obtain a more traditional enriched (3S),(4S) diastereomer product. Interestingly, the use of such enzymatic reaction with the precursor, diester compound (B), was not very successful, largely not specific or specific for the wrong orientation. Thus, unlike the suggestion in WO 2005/283023, here the enzymatic resolution with PLE is advantageously carried out on the lactam of formula (2).

The PLE is preferably introduced into the reaction in an immobilized form. Such form is commercially available. The resolution with PLE is conveniently performed in an aqueous medium of pH of about 7, at a temperature of 35-40 degrees Celsius. The course of the reaction may be monitored by a suitable analytical technique, e.g., by HPLC. After the reaction is complete, the enzyme is removed by filtration, the aqueous reaction mixture is alkalinized and the product is extracted by a suitable water-immiscible organic solvent, while the acid remains in the aqueous phase. After isolation from the organic solvent, the desired trans-(2) is obtained as a substantially pure diastereomer. The “substantially pure diastereomer” should be understood as that the obtained compound corresponding to formula (2) consists of more than 95% of the (3S),(4S) diastereomer, i.e., its diastereomeric purity is higher than 95%.

The substantially pure (3S),(4S) diastereomer of the formula (2), preferably wherein R is methyl or ethyl group, may be isolated in a solid state form by the same process of precipitation from a contrasolvent as indicated above for the trans-racemate. The solid state form is the preferred form of this single diastereomer for its stability and handling properties, and the precipitation process is preferable as it enhances the chemical purity of the crude product. It is an advantage of the overall process that the (3S),(4S) diastereomer of the compound (2) may be obtained in higher than 98% chemical purity and higher than 95% enantiomeric purity.

The compound of the formula (2), preferably the solid state compound of the formula (2), wherein R is C1-C4 alkyl group and preferably is methyl or ethyl group, incl. the single diastereomer thereof, is then converted into compound of formula (1) and preferably into pregabalin of the formula (1) by processes known in the art. Generally the process involves contacting the compound (2) with a strong acid under enhanced temperature. Preferably the process consists of the sequence of three reactions in an one-pot arrangement:

a) hydrolysis of the ester group into a carboxy group;

b) decarboxylation of the resulting lactam-acid (2A) into a pyrrolidone (2B); and

c) acidic hydrolysis of the resulting pyrrolidone compound (2B) into (1).

This sequence is shown below.

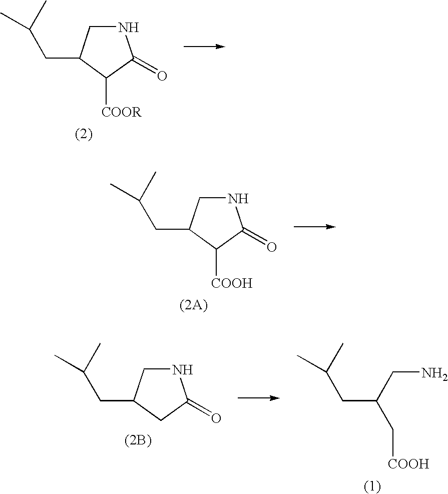

The reaction sequence is also possible to be performed step-wise, i.e., each of the intermediates is isolated (this is suggested in WO 2006/110783). Nevertheless, the above one-pot arrangement is generally preferred.

The useful strong acid may be hydrochloric acid, hydrobromic acid, sulfuric acid, perchloric acid, trifluoroacetic acid, p-toluene sulfonic acid and the like. Hydrochloric acid is the preferred acid. The concentration of the acid may be from 1N to 12 N, the molar amount of the acid may be from 1 to 10 equivalents. The reaction temperature is conveniently from 50 to 150° C. or may be adjusted sequentially (the hydrolysis of the ester group may occur at ambient temperature, then the next steps are performed at elevated temperature).

The trans-racemate of the compound (2) provides a racemate of the compound (1), while the single (3S), (4S) diastereomer of the compound (2) may provide directly the desired (S)-enantiomer of the compound (1), i.e., the pregabalin. The compound (1), and particularly pregabalin, is advantageously isolated from the reaction mixtures by a suitable process, and purified, if necessary.

In summary, the compound (1) may be provided in a relatively high overall yield (when comparing to the starting aldehyde and/or dialkyl malonate) and in a high degree of purity.

The invention is further illustrated by the following non-limiting examples.

### Example 1

### Dimethyl 2-(3-methyl-butylidene)malonate

The mixture of isovaleraldehyde (17.2 g, 21.5 ml, 0.2 Mol), dimethyl malonate (52.8 g, 0.4 Mol) and (S)-proline (2.30 g, 0.02 Mol) in DMSO (70 ml) was stirred overnight at ambient temperature. Afterwards, ethyl acetate (100 ml) was added, and the mixture was extracted by water (3×75 mm). The organic layer was dried over sodium sulfate, filtered and the solvent was evaporated.

The residue was distilled at the oil pump and the dimethyl malonate fraction was removed.

Distillation of the crude product gave 35.37 gram material as an oil. (88%)

### Example 2

### Dimethyl 2-(3-methyl-1-nitromethyl-butyl)malonate

A mixture of Dimethyl 2-(3-methyl-butylidene)malonate (7.7 g, 38.5 mmoles) of nitromethane (2.7 ml, 50 mmoles)_and_DBU (0.75 g, 5 mmoles) in toluene (50 ml) was stirred for two hours at ambient temperature.

Reaction mixture was worked-up by washing with 1N HCl (25 ml) followed by a washing with water (25 ml). The organic phase was dried over sodium sulfate. Evaporation of the solvent gave 10.17 g of residue, which was further evaporated at the oil pump at 60° C. to remove the rests of volatiles.

Slightly yellow oil, 9.91 gram.

### Example 3 (Comparative)

### Hydrogenation of Dimethyl 2-(3-methyl-1-nitromethyl-butyl)malonate

1.0 gram of Dimethyl 2-(3-methyl-1-nitromethyl-butyl)malonate was dissolved in 25 ml acetic acid and 2 ml of water was added. The mixture was hydrogenated with 10% Pd/C catalyst at 1.5 ato for 2 hrs. According to HPLC, starting material has disappeared and the product is mainly the hydroxylactam (5). The hydrogen pressure was raised to 4 ato and the mixture hydrogenated for 2 hrs.

The catalyst was removed by filtration, solution was evaporated, residue triturated with ether/heptane. 170 mg of a solid precipitated. Composition: 74% hydroxylactam, 22% lactam.

### Example 3A (Comparative)

### Hydrogenation of Diethyl 2-(3-methyl-1-nitromethyl-butyl)malonate

3.0 gram of the Diethyl 2-(3-methyl-1-nitromethyl-butyl) malonate was dissolved in 50 ml of acetic acid and 300 mg of Pd/C was added. The mixture was hydrogenated at 3.0 bar for 3 hrs.

The catalyst was filtered off and the solvent evaporated.

HPLC of the oily product indicates that main part is hydroxylactam (5) and some lactam (2) is formed.

### Example 4

### Hydrogenation of Dimethyl 2-(3-methyl-1-nitromethyl-butyl)malonate

1. Hydrogenation resulting in the mixture of (2) and (5)

The hydrogenation experiment was repeated, however now a mixture of acetic acid (15 ml) and MeOH (10 ml) for 1 gram of nitro-compound was used. Amount of 10% Pd/C 100 mg. Pressure 1.5 ato.

After 3 hrs, the pressure was raised to 2 ato. After another 3.5 hrs, starting material had disappeared, however still mixture of mainly hydroxylactam (5) and lactam (2).

The catalyst was filtered out, the mixture was evaporated, dissolved in 20 ml of acetic acid and a new hydrogenation attempt was performed at 100 ato overnight. No improvement in the composition of the reaction mixture.

2. Ra—Ni hydrogenation of the mixture to provide compound (2)

The catalyst was removed by filtration, the residue was concentrated under vacuum and hydrogenated with Ra—Ni at 2.7 ato. After 3 hrs. the mixture was worked up and HPLC showed complete transformation to the lactam compound (2).

### Example 5

### Methyl 4-isobutyl-2-oxo-pyrrolidine-3-carboxylate (compound (2), R=methyl)

5.1 gram of Dimethyl 2-(3-methyl-1-nitromethyl-butyl)malonate was dissolved in MeOH (80 ml). Approx. 500 mg of Ra—Ni that was washed with MeOH (3×) was added and the mixture was hydrogenated in Parr apparatus at 2 bar. After approximately 4 hrs. the HPLC showed that the starting material was gone. The pressure was raised to 5 bar and allowed to react during the night.

The catalyst was filtered through filter disk, the brown colored solution was evaporated, to yield brown oil (3.8 gram). The oil was diluted with small amount of i-propylether, whereby a crystallization starts. After one night the crystals were filtered off and washed with heptane/i-propylether 1:1. This gave 2.58 g, 66% of the title product.

### Example 6

### Methyl 4-isobutyl-2-oxo-pyrrolidine-3-carboxylate (compound (2), R=methyl)

35 gram of Dimethyl 2-(3-methyl-1-nitromethyl-butyl)malonate (0.13 Mol) was dissolved in MeOH (275 ml) and approx. 3 grams of Ra—Ni that was washed with MeOH (3×) was added. The mixture was hydrogenated in Parr apparatus at 2 bar overnight. After HPLC analysis, the pressure was raised to 5 bar and hydrogenated during another night. HPLC showed almost no hydroxylactam.

After filtration of the catalyst, the brown filtrate was concentrated and the residue stirred with a mixture of heptane/isopropylether 1:1 (50 ml). This gave a solid that was filtered with suction and washed with small amounts of solvent mixture.

After drying, 19 gram of off-white material (71%) was obtained.

From the mother liquor another 1.0 gram of material was isolated.

### Example 7

### Enzymatic resolution of Methyl 4-isobutyl-2-oxo-pyrrolidine-3-carboxylate

a) 100 mg of Methyl 4-isobutyl-2-oxo-pyrrolidine-3-carboxylate was dissolved in 5 ml water. A small amount (few mg) of PLE was added. The mixture was stirred magnetically, pH was kept around 7 by adding saturated sodium bicarbonate solution. After one hour HPLC indicated already 27% acid formation.

The mixture was left stirring over the weekend. According to HPLC, 54% acid, 43% lactam ester.

b) Another 500 mg of the Methyl 4-isobutyl-2-oxo-pyrrolidine-3-carboxylate was dissolved in 25 ml of water. Small amount of PLE was added. The pH was kept at 7, temp was kept at 35 C. pH was regulated with 0.2 N NaOH. The mixture was stirred during weekend.

HPLC: 59% acid, 39% lactam ester.

### Work-Up

Combined reaction mixtures were filtered through Celite. The solution was made basic with satd. sodium bicarbonate and extracted twice with dichloromethane. Combined organic layers were dried over sodiumsulfate. Water layer was acidified with 2N sulfuric acid, saturated by NaCl and extracted with dichloromethane. Combined organic layers were dried over sodiumsulfate.

Work-up of the both dichloromethane extracts gave 0.23 gram of the lactam ester and 0.41 gram of the lactam acid, resp.

### Verification of Conformation

Both products were hydrolysed with 6N HCl at reflux overnight.

Both reaction mixtures were evaporated to dryness as good as possible. Upon trituration with diethylether, the product derived from the lactam ester gave a colourless solid. The HPLC confirmed the structure of pregabalin. Optical purity after Marfey derivatisation: S-Pregabalin: R-Pregabalin 97.34:2.66

The lactam-acid derived product exhibited slower crystallization. After derivatisation with Marfey's reagent S-Pregabalin:R-Pregabalin 22:78

### Example 8 (Comparative)

Prior art process for Dimethyl 2-(3-methyl-1-nitromethyl-butyl)malonate

Step 1—Preparation of nitroalkene (14)

According to Ballini and Bosica: J. Org. Chem., 1997, 62, 425, the reaction between aldehyde and nitromethane can be best performed under aqueous conditions and by use of a phase transfer catalyst.

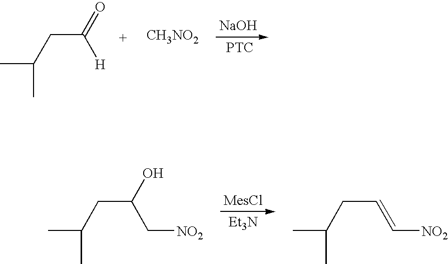

Step 1A

The mixture of isovaleraldehyde (4.3 g, 50 mmoles), nitromethane (3.5 g, 50 mmoles), sodium hydroxide (50 mg, 1.25 mmoles) and bezyltrimethylammoniumchloride (0.93 g, 5 mmoles) was stirred in water (50 ml) for one night. The mixture was extracted with EtOAc

After work-up 5.75 of crude material (nitroaldol) was obtained.

Step 1B

The nitroaldol (6.60 g, 44.9 mmoles) was dissolved in dichloromethane (25 ml). 5.75 g (50 mmoles) of methane sulfonylchloride was added, followed by Et₃N (6.00 g, 6 mmoles). The mixture was stirred in an ice-water bath for one hour.

The dichloromethane solution was washed with water, dried over sodiumsulfate, and evaporated. The residue was chromatographed over 240 g of silica, elution with EtOAc-heptane 1:8.

1.29 g of pure nitroalkene was obtained.

Step 2—Addition of dimethyl malonate to the nitroalkene.

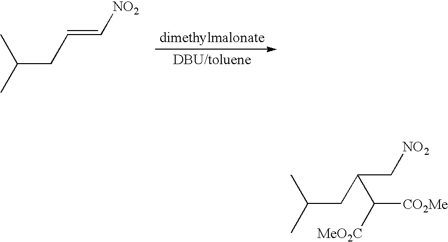

Nitroalkene (4.45 g, 34.5 mmoles) was dissolved in toluene (20 ml), dimethyl malonate (6.6 g, 50 mmoles) was added, followed by DBU (532 mg, 3.5 mmoles). The reaction mixture was stirred at ambient temperature overnight.

The solution was diluted with 25 ml of EtOAc, washed with 1N HCl, water, and dried over sodiumsulfate.

The solvent was evaporated and the excess of malonate was removed with oil pump.

Yield: Red oil, 5.0 gram

### Example 9—Comparative

Ex 5 of WO 2006/110783:

A) Verification of the Example with Racemic Starting Material

1.4 gram of racemic nitro-dimethyl malonate (B) was dissolved in 15 ml MeOH. 0.5 gram NiCl₂ hexahydrate was added. The mixture was cooled in ice-water and 0.33 gram of sodium borohydride was added. After 3 hrs, the reaction mixture was analysed by HPLC showing a mixture of hydroxy-lactam (5) and lactam (2) in a ratio 39:41. After another two hours, no change in the ratio.

Another 100 mg of sodiumborohydride was added and the mixture was stirred for two more hours. Saturated aqueous ammonium chloride was added and the mixture was extracted with dichloromethane. The extract was dried with sodium sulfate and evaporated yielding. 0.78 gram of slightly greenish oil. According to HPLC, the oil comprises 34.5% of the hydroxylactam (5) and 57% of lactamester (2).

B) Verification with the Optically Active Starting Material

1.4 gram of the (R)-dimethylnitromalonate (B) was dissolved in 14 ml MeOH. 500 mg powdered NICl₂.6H₂O was added. The mixture was stirred with ice-water cooling and 330 mg of fresh sodium borohydride was added. Stirring was continued for 6 hrs.

After 3.5 hrs, the mixture was analysed by HPLC (mainly hydroxylactam, still some starting material). Another 300 mg sodium borohydride was added in portions and the mixture was stirred overnight. HPLC confirmed that the starting material was gone. The mixture was worked-up by ammonium chloride solution and by extraction with dichloromethane.

Obtained 0.91 gram of a residue. HPLC: Hydroxylactam (5) 34% and lactamester (2) 50%.

Each of the patents, patent applications, and journal articles mentioned above are incorporated herein by reference. The invention having been described it will be obvious that the same may be varied in many ways and all such modifications are contemplated as being within the scope of the invention as defined by the following claims.

I claim:

1. A process which comprises:
(i) reacting isovaleraldehyde with an excess of a dialkyl malonate of the formula (10) 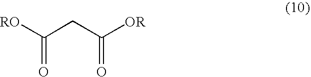
wherein R is a C1-C4 alkyl group, in the presence of a base reagent having a combination of a secondary amine and a carboxylic acid to obtain a compound of formula (A) 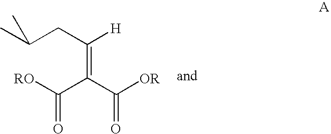
(ii) reacting the compound of formula (A) with nitromethane, under conditions of Michael addition, to yield the compound of formula (B) 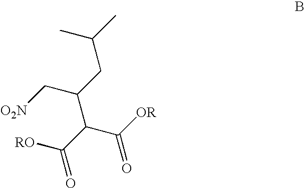
wherein R is as previously defined.

2. The process according to claim 1, wherein said base reagent is an iminoacid compound.

3. The process according to claim 2, wherein said base reagent is proline.

4. The process according to claim 1, wherein said compound of formula (A) produced in step (i) is isolated from the reaction prior to being reacted in said step (ii), wherein said isolating of compound (A) achieves a dialkyl malonate content of less than 1%.

5. The process according to claim 4, wherein said dialkyl malonate content is less than 0.5%.

6. The process according to claim 1, wherein said step (ii) proceeds in the presence of a catalytic and sub-stoichiometric amount of a base, preferably selected from 8-diazabicyclo[5.4.0]undec-7-ene (DBU), 1,4-diazabicyclo[2.2.2]octane (DABCO), 1,5-diazabicyclo(4:3:0)non-5-ene (DBN), and 4-dimethyl-aminopyridine (DMAP).

7. The process according to claims 1, which further comprises converting said compound of formula (B) into pregabalin.

8. A process which comprises subjecting a compound of formula (B) 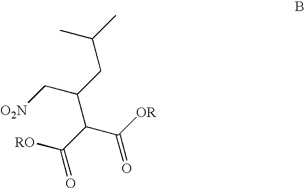
to reductive cyclization by hydrogen to form a compound of formula (2) 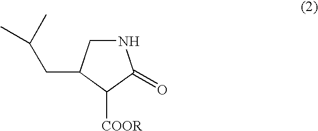
wherein R is a C1-C4 alkyl group and wherein said reductive cyclization is carried out in the presence of Raney-nickel.

9. The process according to claim 8, wherein said reductive cyclization is carried out under greater than atmospheric pressure of hydrogen.

10. The process according to claim 8, wherein R is methyl or ethyl.

11. The process according to claim 8, wherein the compound (2) is obtained essentially free from the compound of formula (5) 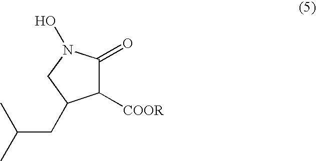
wherein R is a C1-C4 alkyl group.

12. The process according to claim 8, wherein said compound of formula (B) is a racemate and wherein said process further comprises resolving said compound of formula (2) to form an enriched single diastereomer.

13. The process according to claim 12, wherein said resolution is carried out by a reaction with pig liver esterase.

14. The process according to claim 8, wherein the compound (2) is obtained in a solid crystalline state.

15. The process according to claim 8, which further comprises converting said compound of formula (2) into pregabalin.

16. The process according to claim 15, wherein said conversion comprises obtaining the compound of formula (2) substantially as the (3S),(4S) diastereomer and treating said diastereomer of compound (2) with a strong acid under reactive conditions to form pregabalin.

17. A process for resolution, which comprises reacting a mixture which contains (3S),(4S) and (3R),(4R) diastereomers of the formula (2) 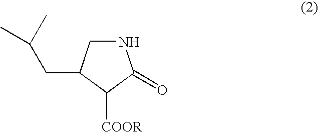
wherein R is a C1-C4 alkyl group, with an enzyme to obtain an enriched (3S),(4S)-diastereomer-containing mixture of the compound of formula (2).

18. The resolution process according to claim 17, wherein said enzyme is pig liver esterase and the reaction selectively hydrolyzes the ester group in the 3R position to form a (3R),(4R) acid of formula (2a)

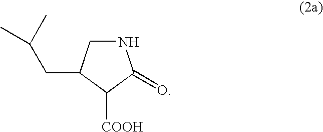

19. The resolution process according to claim 18, which further comprises isolating the enriched (3S)(4S) compound of formula (2) in a crystalline state.

20. The resolution process according to claim 18, which further comprises treating the (3S),(4S)-diastereomer of the compound (2) with a strong acid under reactive conditions to form pregabalin.

21. A process which comprises:
(a) subjecting a compound of formula (B) 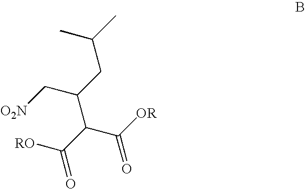
to reductive cyclization by hydrogen to form a compound of formula (2) 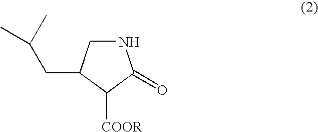
wherein R is a C1-C4 alkyl group and wherein said reductive cyclization is carried out in the presence of a hydrogen catalyst and under greater than atmospheric pressure of hydrogen;
(b) monitoring the reaction for the presence of the compound of formula (5) 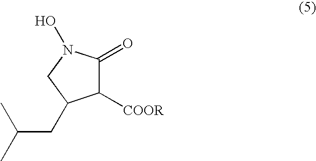
wherein R is a C1-C4 alkyl; and
(c) terminating the reaction when the content of (5) is less than a predetermined amount.

22. The process according to claim 21, wherein said predetermined amount of formula (5) is less than 1%.

23. A compound of formula (2) 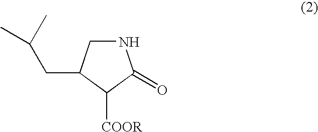
in a crystalline form, wherein R is a C1-C4 alkyl group.

24. The compound according to claim 23, wherein said compound is 3,4-trans compound and is substantially the single (3S),(4S)-diastereomer; and wherein R is methyl or ethyl.

25. The compound according to claim 24, wherein said compound has a chemical purity of at least 95%.

26. A process for precipitating the compound (2), which comprises contacting a solution which contains a compound of formula (2) 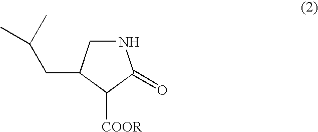
wherein R is a C1-C4 alkyl, with an antisolvent to precipitate said compound of formula (2) as a solid material.

27. The process according to claim 26, wherein the antisolvent is a dialkylether, a C5-C10 hydrocarbon, or mixtures thereof.

28. The process according to claim 27, wherein said antisolvent is selected from the group consisting of di-isopropyl ether, methyl isopropylether, hexane, heptane, benzene, toluene, and mixtures thereof.

29. The process according to claim 26, wherein said solid material is a crystalline solid material.

30. A compound of formula (5) 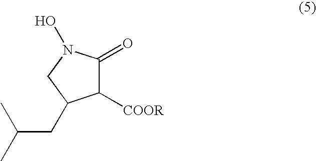
wherein R is a C1-C4 alkyl group, in an isolated form having a purity of at least 60%.
